(19) United States
(12) Patent Application Publication
(10) Pub. No.: US 2008/0144909 A1
(43) Pub. Date: Jun. 19, 2008

(54) Analysis of Pulmonary Nodules from Ct Scans Using the Contrast Agent Enhancement as a Function of Distance to the Boundary of the Nodule

(75) Inventors: Rafael Wiemker, Kisdorf (DE); Dag Wormanns, Muenster (DE); Thomas Blaffert, Hamburg (DE); Thomas Buelow, Grosshansdorf (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/815,601

(22) PCT Filed: Feb. 3, 2006
(86) PCT No.: PCT/IB06/50361

§ 371 Date: Aug. 6, 2007

(30) Foreign Application Priority Data
Feb. 11, 2005 (EP) 05101010.6

(51) Int. Cl. G06K 9/62; G06K 9/68; A61B 6/00; A61B 5/055; G06T 7/60

(52) U.S. Cl. 382/131; 382/132; 382/225; 378/21; 324/312; 600/425

(57) ABSTRACT

For differential diagnosis of pulmonary nodules, a certain fraction of malignant nodules do not exhibit significant enhancement when averaged over the whole nodule volume. According to an exemplary embodiment of the present invention, not only a single averaged contrast enhancement number is determined, but an enhancement curve for each nodule, showing the enhancement as a function of distance to boundary of the nodule. This may provide for an improved differential diagnosis.

The present invention relates to the field of examination of an object of interest. In particular, the invention relates to an examination apparatus for examination of an object of interest, an image processing device, a method of examining an object of interest with an examination apparatus, a computer-readable medium and a program element.

Evaluation of a solitary pulmonary nodule remains a substantial and costly challenge in modern medicine. Approximately 50% of indeterminate lung nodules for which surgery is performed for diagnosis are benign. Hospitalization for surgical removal of a nodule involves significant expenses.

For differential diagnosis of pulmonary nodules into malignant versus benign, assessment of contrast enhancement at chest CT scans after administration of contrast agent is used. However, a certain fraction of malignant nodules do not exhibit significant enhancement when averaged over the whole nodule volume.

It would be desirable to provide for a means that diagnostic radiologists can use to substantially reduce the percentage of benign nodules for which surgery is performed, thus resulting in an improved examination of a nodule.

In accordance with an exemplary embodiment of the present invention, the above desire may be met by an examination apparatus for examination of an object of interest, the examination apparatus comprising a determination unit adapted for determining enhancement characteristics of the object of interest and a comparison unit adapted for comparing the enhancement characteristics with a reference, resulting in a comparison result, wherein the enhancement characteristics is a function of a distance to a boundary of the object of interest.

Thus, an examination apparatus is provided which does not only provide a single averaged contrast enhancement number, but may provide an enhancement curve for each single object of interest, showing the enhancement of that object as a function of a distance to the boundary of the object of interest.

Advantageously, this may lead to an improved examination of the object of interest and therefore to an improved differential diagnosis.

According to another exemplary embodiment of the present invention, the examination apparatus further comprises a segmentation unit adapted for performing a two-dimensional or a three-dimensional segmentation of image data of the object of interest, wherein the determination unit is adapted for determining the enhancement characteristics of the object of interest on the basis of the segmentation.

Advantageously, according to this exemplary embodiment of the present invention, a multi-dimensional segmentation of the image data may be performed, for example, by means of an automated unsupervised computer procedure. Thus, the object of interest may be isolated from the surrounding materials and may then be further examined.

According to another exemplary embodiment of the present invention, the determination unit is further adapted for determining a centre of the object of interest on the basis of a distance transformation procedure, wherein the centre of the object of interest (7) is used as a starting point for the enhanced characteristics determination.

The application of a distance transformation procedure, according to this exemplary embodiment of the present invention, may lead to a precise and well-defined centre identification, even if image data is compared which has been acquired at different points in time.

According to another exemplary embodiment of the present invention, the object of interest is a nodule and the comparison result is a measure for one of a malignancy and an inflammation of the nodule.

Advantageously, a predetermined threshold value may be used and an alarm may be triggered or the user may be otherwise notified of the malignancy or inflammation of the nodule, if the predetermined threshold value is exceeded. This may allow for a fully automated diagnosis.

According to another exemplary embodiment of the present invention, the examination apparatus further comprises an interpolation unit for interpolating the image data to isotropic resolution.

Advantageously, this may yield to a projection of the image data onto an isotropic lattice or grid, which may yield to an improved standardization of the image data.

According to another exemplary embodiment of the present invention, the segmentation unit is further adapted for performing a removal of lung wall tissue from the image data of the object of interest. Advantageously, this may yield to an improved segmentation.

According to another exemplary embodiment of the present invention, the examination apparatus is one of a CT (computed tomography) scanner system, an MRI (magneto resonance imaging) scanner system, PET (positron emission tomography) scanner system, SPECT (single photon emission computerized tomography) scanner system, and an ultrasound imaging system.

Advantageously, by using different imaging systems, different enhancement characteristics of the object of interest may be determined, thus leading to an improved differential diagnosis.

Furthermore, the examination comprises, according to another exemplary embodiment, an electromagnetic radiation source adapted to emit electromagnetic radiation to the object of interest and a collimator arranged between the electromagnetic source and detecting elements, the collimator being adapted to collimate an electromagnetic radiation beam emitted by the electromagnetic radiation source.

Furthermore, according to another exemplary embodiment of the present invention, the electromagnetic radiation source is a polychromatic x-ray source, wherein the electromagnetic radiation source moves along a helical path around the object of interest and wherein the beam has a fan-beam geometry.

The application of a polychromatic x-ray source may be advantageous, since polychromatic x-rays are easy to generate and provide a high photon flux.

According to another exemplary embodiment of the present invention, an image processing device for examining an object of interest is provided, the image processing device comprising a memory for storing image data of an object of interest and an image processor adapted for performing a determination of enhancement characteristics of the object of interest and a comparison of the enhancement characteristics with a reference, resulting in a comparison result, wherein the enhancement characteristics is a function of a distance to a boundary of the object of interest.

Advantageously, this may provide for an image processing device which is adapted for determining an enhancement curve for each object of interest, showing the enhancement as a function of distance to boundary (or centre) of the object of interest, thus resulting in an improved differential diagnosis.

According to another exemplary embodiment of the present invention, a method of examining an object of interest with an examination apparatus is disclosed, the method comprising the steps of determining enhancement characteristics of the object of interest and comparing the enhancement characteristics with a reference, resulting in a comparison result, wherein the enhancement characteristics is a function of a distance to a boundary of the object of interest.

The present invention also relates to a computer-readable medium and to a program element of examining an object of interest, which is stored on the computer-readable medium. The program element is adapted to carry out the steps of determining enhancement characteristics of the object of interest and comparing the enhancement characteristics with a reference, resulting in a comparison result, when being executed by a processor. The program element may be part of, for example, a CSCT scanner system. The program element, according to an exemplary embodiment of the present invention, may preferably be loaded into working memories of a data processor. The data processor may thus be equipped to carry out exemplary embodiments of the methods of the present invention. The computer program may be written in any suitable programming language, such as, for example, C++ and may be stored on a computer-readable medium, such as CD-ROM. Also, the computer program may be available from a network, such as the WorldWideWeb, from which it may be downloaded into image processing units or processors, or any suitable computers.

An aspect of the present invention may be that the enhancement characteristics of a pulmonary nodule is determined as a function of a distance of a group of voxels (of the nodule) to the boundary of the nodule with the center of the nodule (which is a single voxel) as a starting point. This may provide for an improved differential diagnosis, since the centre of the nodule may be, according to an aspect of the present invention, determined with high accuracy.

These and other aspects of the present invention will become apparent from and elucidated with reference to the embodiments described hereinafter.

Exemplary embodiments of the present invention will be described in the following, with reference to the following drawings:

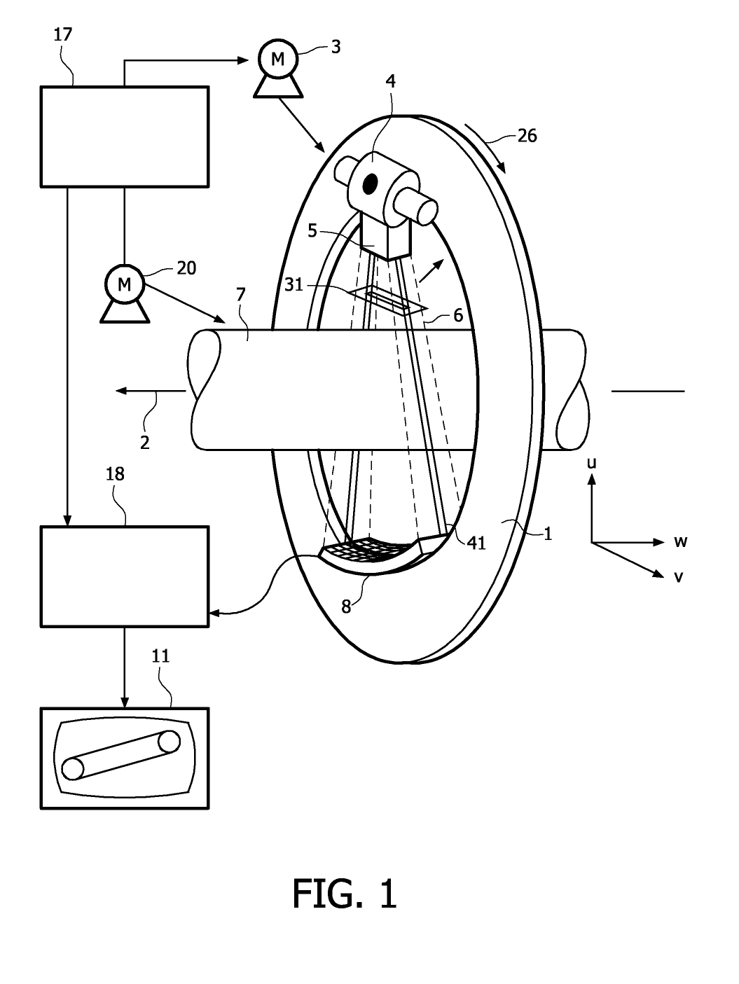

FIG. 1 shows a simplified schematic representation of an embodiment of a CSCT scanner according to the present invention.

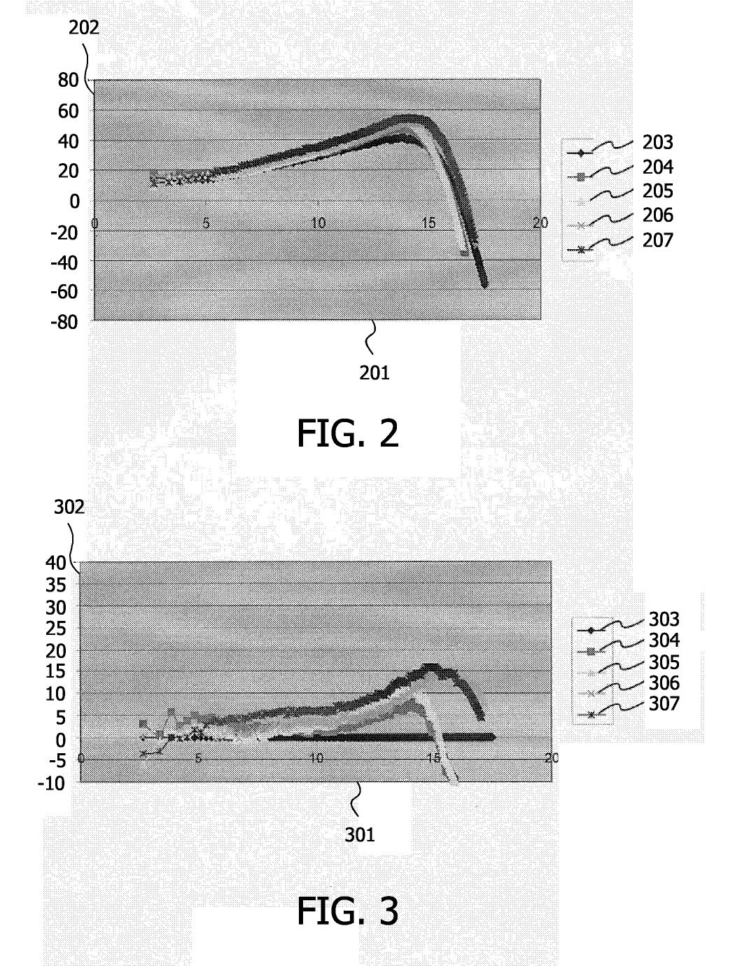

FIG. 2 shows exemplary enhancement-scans of a nodule as a function of effective diameter.

FIG. 3 shows a difference between the exemplary enhancement-scans of FIG. 2 and an unenhanced scan.

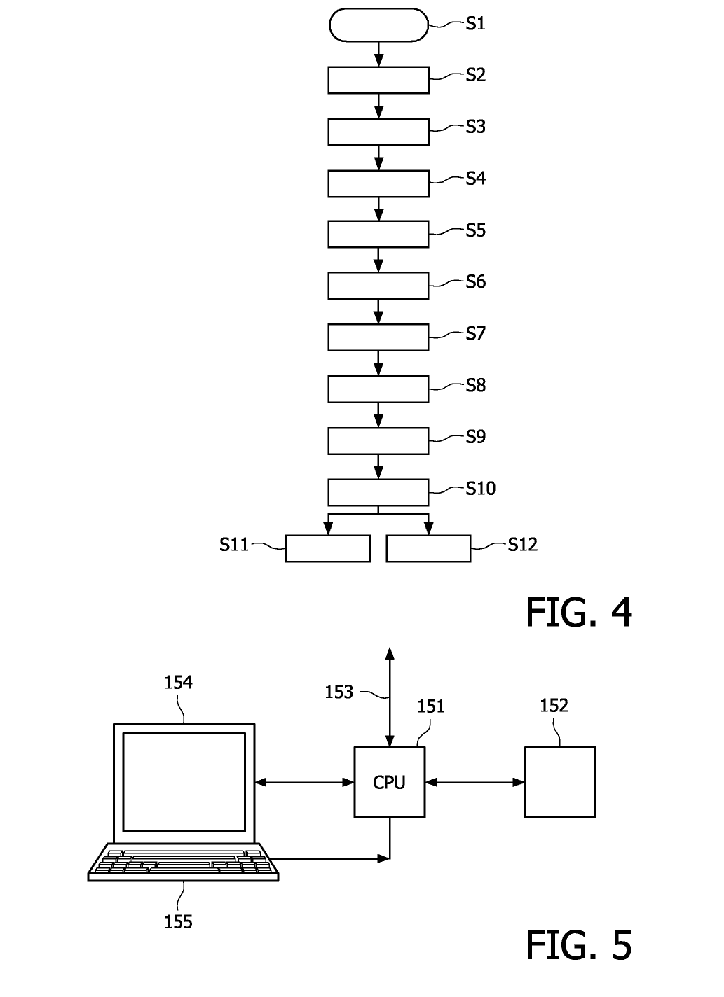

FIG. 4 shows a flow-chart of an exemplary embodiment of a method of examining an object of interest with an examination apparatus according to the present invention.

FIG. 5 shows an exemplary embodiment of an image processing device according to the present invention, for executing an exemplary embodiment of a method in accordance with the present invention.

The illustration in the drawings is schematically. In different drawings, similar or identical elements are provided with the same reference numerals.

With reference to an exemplary embodiment, the present invention will be described for the application in medical imaging to detect malignant or inflammated nodules. However, it should be noted that the present invention is not limited to the application in the field of medical imaging, but may also be used in applications such as baggage inspection, material testing and material science.

The scanner depicted in FIG. 1 is a fan-beam CSCT scanner. The CSCT scanner depicted in FIG. 1 comprises a gantry 1, which is rotatable around a rotational axis 2. The gantry 1 is driven by means of a motor 3. Reference numeral 4 designates a source of radiation, such as an x-ray source, which, according to an aspect of the present invention, emits a polychromatic radiation beam.

Reference numeral 5 designates an aperture system which forms a radiation beam emitted from the radiation source 4 to a radiation beam 6. After emitting the radiation beam 6, the beam may be guided through a slit collimator 31 to form a primary fan-beam 41 impinging on an object 7 to be located in an object region.

The fan-beam 41 is now directed such that it penetrates the object 7 arranged in the centre of the gantry 1, i.e. in an examination region of the CSCT scanner and impinges onto the detector 8. As may be taken from FIG. 1, the detector 8 is arranged on the gantry 1 opposite the source of radiation 4, such that the surface of the detector 8 is covered by the fan-beam 41. The detector 8 depicted in FIG. 1 comprises a plurality of detector elements.

During a scan of the object of interest 7, the source of radiation 4, the aperture system 5 and detector 8 are rotated along the gantry 1 in the direction indicated by arrow 16. For rotation of the gantry 1 with the source of radiation 4, the aperture system and the detector 8, the motor 3 is connected to a motor control unit 17, which is connected to a determination unit 18.

During a scan, the radiation detector 8 is sampled at predetermined time intervals. Sampling results read from the radiation detector 8 are electrical signals, i.e. processed and represent radiation intensity, which may be referred to as projection in the following. A whole data set of a whole scan of an object of interest therefore consists of a plurality of projections where the number of projections corresponds to the time interval with which the radiation detector 8 is sampled. A plurality of projection together may also be referred to as volumetric data. Furthermore, the volumetric data may also comprise electrocardiogram data.

In FIG. 1, the object of interest is disposed on a conveyor belt 19. During the scan of the object of interest 7, while the gantry 1 rotates around the patient 7, the conveyor belt 19 displays the object of interest 7 along a direction parallel to the rotational axis 2 of the gantry 1. By this, the object of interest 7 is scanned along a helical scan path. The conveyor belt 19 may also be stopped during the scans. Instead of providing a conveyor belt 19, for example, in medical applications, where the object of interest 7 is a patient, a movable table may be used. However, it should be noted that in all of the described cases it is also possible to perform a circular scan, where there is no displacement in a direction parallel to the rotational axis 2, but only the rotation of the gantry 1 around the rotational axis 2.

The detector 8 is connected to the determination unit 18. The determination unit 18 receives the detection result, i.e. the read-outs from the detector element of the detector 8, and determines a scanning result on the basis of the read-outs. The detector elements of the detector 8 may be adapted to measure the attenuation caused to the fan-beam 6 by the object of interest 7 or the energy and intensity of x-rays coherently scattered from an object point of the object of interest 7 with an energy inside or certain energy interval. Furthermore, the determination unit 18 communicates with the motor control unit 17 in order to coordinate the movement of the gantry 1 with motor 3 and 20 or with a conveyor belt (not shown in FIG. 1).

The determination unit 18 may be adapted for reconstructing an image from read-outs of the detector 8. The image generated by the determination unit 18 may be output to a display 11.

The determination unit 18 which may be realized by an image processing device may also be adapted to perform a determination of enhancement characteristics of the object of interest 7 and a comparison of the enhancement characteristics with a reference, resulting in a comparison result, wherein the enhancement characteristics is a function of a distance to a centre of the object of interest 7. Furthermore, the image processing device may further be adapted for performing a multi-dimensional segmentation of the image data of the object of interest, wherein the enhancement characteristics of the object of interest is determined on the basis of the segmentation.

Furthermore, as may be taken from FIG. 1, the determination unit 18 may be connected to a loudspeaker to, for example, automatically output an alarm.

FIG. 2 shows exemplary enhancement-scans of a nodule as a function of effective diameter. The curves depicted in FIG. 2 are taken at different points in time. The first curve 203 which is the native curve reflects the unenhanced scan before application of a contrast agent. The second curve 204 represents data acquired 60 seconds after application of contrast agent, the third curve 205 represents data acquired 120 seconds after application, the fourth curve 206 represents data acquired 180 seconds after application and the fifth curve 207 represents data acquired at 240 seconds after application of contrast agent.

The horizontal axis 201 represents the effective diameter of the nodule. By effective diameter we mean the volume-equivalent diameter, i.e., the diameter of an ideal sphere having the same volume. The vertical axis 202 represents the mean Hounsfield values as a function of the distance to a boundary of the nodule. For each nodule and time point, the mean Hounsfield value is computed first for the voxels most distant to the nodule boundary (central to the nodule), then the distance to the boundary is lowered continuously and more and more voxels are taken into account, yielding a mean Hounsfield curve as a function of cumulative core-to-rim voxels, i.e. of cumulative volume.

In order to allow comparability between native and contrasted scans, which may yield slightly different nodule outlines and may have different voxel spacings, the mean Hounsfield curve is given as a function of cumulative volume (from the inner most to outer most voxels) or effective diameter.

It should be noted that the voxels for which a respective mean Hounsfield value is determined, may not be positioned on a concentric circle (or sphere, in three dimensions) around the centre of the nodule, since they all have the same distance from the nodule boundary. Rather, these voxels lie in layers parallel to the boundary surface. Thus, the nodules for which a respective mean Hounsfield value is determined, may lie on a surface which reflects the outer shape of the nodule. After calculation of a certain mean Hounsfield value, a next layer of voxels (i.e. the group of voxels which lies a step further from the center towards the nodule boundary) is identified and the respective mean Hounsfield value determined. Repeating this operation yields to the mean Hounsfield curve depicted in FIG. 2.

The curves 303-307 of FIG. 3 show a difference between the scans 203-207 of FIG. 2 and the native curve 203. As may be seen from FIG. 3, the differential curves depicted in FIG. 3 show enhancement at the outer parts of the nodule. Curve 307, which represents nodule image data acquired at 240 seconds after application of the contrast agent subtracted by the nodule image data of the unenhanced scan 203 (of FIG. 2), shows a significant enhancement (>15 Hounsfield units) and is therefore considered as representing a malignant nodule.

The method starts at step S1 with an administration of contrast agent to the patient. Then, at step S2, the CT scan starts and image data is acquired. After acquisition of the image data of the object of interest, a three-dimensional segmentation of the nodule is performed in step S3. For each of the CT scans at different time points, the nodule is three-dimensionally segmented by an unsupervised computer procedure. After that, in step S4, the data volumes or volumetric data is interpolated to isotropic resolution. This may yield to a standardization of the image data. According to an aspect of the present invention, the segmentation may include nodule tissue above −400 Hounsfield units.

Then, in step S5, lung wall tissue is removed morphologically, if necessary. In step S6 then, attached vessels are cut off morphologically at the thinnest connection. Thus, the nodule is completely isolated from the surrounding tissue and may in the following be analyzed.

In the next step (S7) the centre of the nodule is determined on the basis of a distance transformation procedure, wherein the centre of the nodule is used as a starting point of the enhancement characteristics determination. For each nodule and time point, the mean Hounsfield value is computed first for the voxels most distant to the nodule boundary (central to the nodule), then the distance to the boundary is lowered continuously and more and more voxels are taken into account, yielding a mean Hounsfield curve as a function of cumulative core-to-rim voxels (step S8).

In order to allow comparability between native and contrasted scans, which may yield different nodule outlines and may have different voxel spacings, the mean Hounsfield curve is determined as a function of cumulative volume (from inner most to outer most voxels). These curves for the contrasted scans are then compared to the respective curves of the unenhanced scan, i.e. the curve of the unenhanced scan is subjected from the contrasted curves, in step S9. This yields a comparison result which is then, in step S10, compared to a predetermined threshold criteria. If the enhancement curves show considerable enhancement, for example more than 15 Hounsfield units, over the whole curve or in parts of the curve, the nodule is considered potentially malignant or inflammated and the method continues with step S11 by, for example, notifying a user or triggering an alarm. Otherwise, if the threshold criteria (of 15 Hounsfield units) is not met, the nodule is considered as being benign, in which case the method continues with step S12, where it ends.

It should be noted that the threshold criteria may be pre-set by a user or automatically depending on the desired level of sensitivity.

FIG. 5 depicts an exemplary embodiment of an image processing device according to the present invention for executing an exemplary embodiment of the method in accordance with the present invention. The image processing device depicted in FIG. 5 comprises a central processing unit (CPU) image processor 151 connected to a memory 152 for storing an image depicting an object of interest. The data processor 151 may be connected to a plurality of input/output network or diagnosis devices, such as a CSCT apparatus. The data processor may furthermore be connected to a display device 154, for example, a computer monitor, for displaying information or an image computed or adapted in an image processor 151. An operator or user may interact with the image processor 151 via a keyboard 155 and/or other output devices, which are not depicted in FIG. 5.

Furthermore, via the bus system 153, it may also be possible to connect the image processing and control processor 151 to, for example, a motion monitor, which monitors a motion of the object of interest. In case, for example, a lung of a patient is imaged, the motion sensor may be an exhalation sensor. In case, the heart is imaged, the motion sensor may be an electrocardiogram.

The examination of an object of interest according to the present invention may allow for a visualization of contrast enhancement which result in a reduction of falls negative results of dynamic CT or other scanner systems, such as MRI (magneto resonance imaging) scanner systems, PET (positron emission tomography) scanner systems, SPECT (single photon emission computerized tomography) scanner systems or ultrasound imaging systems, of pulmonary nodules. Therefore, a diagnostic tool for differential diagnosis between malignant and benign lesions may be provided.

Exemplary embodiments of the invention may be sold as a software option to CT scanner console, imaging workstations (extended brilliance workspace, view forum), and PACS workstations.

It should be noted that the term “comprising” does not exclude other elements or steps and the “a” or “an” does not exclude a plurality and that a single processor or system may fulfill the functions of several means or units recited in the claims. Also elements described in association with different embodiments may be combined.

It should also be noted, that any reference signs in the claims shall not be construed as limiting the scope of the claims.

1. An examination apparatus for examination of an object of interest, the examination apparatus comprising:
a determination unit adapted for determining enhancement characteristics of the object of interest;
a comparison unit adapted for comparing the enhancement characteristics with a reference, resulting in a comparison result;
wherein the enhancement characteristics is a function of a distance to a boundary of the object of interest.

2. The examination apparatus of claim 1, further comprising:
a segmentation unit adapted for performing a two-dimensional or a three-dimensional segmentation of image data of the object of interest;
wherein the determination unit is adapted for determining the enhancement characteristics of the object of interest on the basis of the segmentation.

3. The examination apparatus of claim 1,
wherein the determination unit is further adapted for determining a centre of the object of interest on the basis of a distance transformation procedure; and
wherein the centre of the object of interest is used as a starting point for the enhanced characteristics determination.

4. The examination apparatus of claim 1,
wherein the object of interest is a nodule; and
wherein the comparison result is a measure for one of a malignancy and an inflammation of the nodule.

5. The examination apparatus of claim 1,
further comprising an interpolation unit for interpolating the image data to isotropic resolution.

6. The examination apparatus of claim 2,
wherein the segmentation unit is further adapted for performing a removal of lung wall tissue from the image data of the object of interest.

7. The examination apparatus of claim 1,
wherein the examination apparatus is one of a CT scanner system, an MRI scanner system, a PET scanner system, a SPECT scanner system, and an ultrasound imaging system.

8. The examination apparatus according to claim 1, comprising an electromagnetic radiation source adapted to emit electromagnetic radiation to the object of interest and comprising a collimator arranged between the electromagnetic radiation source and the detecting elements, the collimator being adapted to collimate an electromagnetic radiation beam emitted by the electromagnetic radiation source.

9. The examination apparatus of claim 1,
wherein the electromagnetic radiation source is a polychromatic x-ray source;
wherein the electromagnetic radiation source moves along a helical path around the object of interest; and
wherein the beam has a fan-beam geometry.

10. An image processing device for examining an object of interest, the image processing device comprising:
a memory for storing image data of an object of interest;
an image processor adapted for performing the following operation:
determining enhancement characteristics of the object of interest;
comparing the enhancement characteristics with a reference, resulting in a comparison result;
wherein the enhancement characteristics is a function of a distance to a boundary of the object of interest.

11. A method of examining an object of interest with an examination apparatus, the method comprising the steps of:
determining enhancement characteristics of the object of interest;
comparing the enhancement characteristics with a reference, resulting in a comparison result;
wherein the enhancement characteristics is a function of a distance to a boundary of the object of interest.

12. A computer-readable medium, in which a computer program of examining an object of interest with an examination apparatus is stored, which, when being executed by a processor, is adapted to carry out the steps of:
determining enhancement characteristics of the object of interest;
comparing the enhancement characteristics with a reference, resulting in a comparison result;
wherein the enhancement characteristics is a function of a distance to a boundary of the object of interest.

13. A program element of examining an object of interest, which, when being executed by a processor, is adapted to carry out the steps of
determining enhancement characteristics of the object of interest;
comparing the enhancement characteristics with a reference, resulting in a comparison result;
wherein the enhancement characteristics is a function of a distance to a boundary of the object of interest.
